(19) United States
(12) Patent Application Publication
(10) Pub. No.: US 2024/0245321 A1
(43) Pub. Date: Jul. 25, 2024

(54) Gait Analysis Device and Method for Providing Service Based on Gait Analysis

(71) Applicants: Hyun-Sang Cho, Seoul (KR); CiKLux Inc., Seoul (KR)

(72) Inventors: Hyun-Sang CHO, Seoul (KR); Sung Eun KIM, Seoul (KR)

(21) Appl. No.: 18/419,202

(22) Filed: Jan. 22, 2024

(30) Foreign Application Priority Data
Jan. 25, 2023 (KR) 10-2023-0009507

(51) Int. Cl. A61B 5/11; A61B 5/00; G16H 50/20

(52) U.S. Cl. CPC A61B 5/112; A61B 5/4824; A61B 5/6895; A61B 5/7285; A61B 5/7435; G16H 50/20

(57) ABSTRACT

The present disclosure proposes a specific technical solution that realizes an environment in which quantitative/objective gait analysis results can be obtained in the form of lifelog data without the need for complex and expensive equipment, thereby enabling the provision of various services (e.g., disease diagnosis services, home training services, content-linked services, and the like) based on gait analysis results, such as enabling disease prediction in the concept of precision medicine.

### CROSS-REFERENCE TO RELATED APPLICATION(S)

This application is based on and claims priority under 35 U.S.C. 119 to Korean Patent Application No. 10-2023-0009507, filed on Jan. 25, 2023, in the Korean Intellectual Property Office, the disclosure of which is herein incorporated by reference in its entirety.

### BACKGROUND OF THE INVENTION

### Field of the Invention

The present disclosure relates to a technology for analyzing the gait of a person (a user).

In particular, the present disclosure relates to a technology for quantitatively and objectively analyzing gait on an existing treadmill by using a sensor that can be attached to the treadmill in a simple manner without modification.

### Description of the Prior Art

Walking, as one of fundamental activities that humans (persons) have been doing for a long time, is used as a popular form of exercise (a fundamental training element) with various physiological benefits. In addition, gait has different characteristics for each person depending on lifestyle, physical characteristics, psychological characteristics, and health conditions, and is thus analyzed and used to estimate the different characteristics.

In particular, kinematic gait stability is known to be correlated with gait speed, muscle strength, and joint range of motion.

That is, many orthopedic or neurological diseases are likely to cause an abnormal gait, and conversely, incorrect gait movements may lead to diseases in the brain, body structures, joints, muscles, and so on. Thus, gait information is recognized as important information that doctors need to consider for medical diagnosis.

There have been cases in which gait information is used to develop algorithms that extract gait features, such as balance, speed, posture, and step length, for patients with specific diseases to determine the severity of the diseases, or is used to track the progress of treatment for patients with degenerative brain diseases

To achieve this, it is necessary to accurately analyze the gait of the patient (person) to extract and obtain accurate gait features and gait information.

Current gait analysis methods in use include the “Timed Up & Go (TUG)” test, in which a person walks a predetermined distance under an examiner's instructions and the result is evaluated, and vision-based analysis, which uses visual markers and foot pressure sensors.

In the case of the “Timed Up & Go (TUG)” test, the accuracy of gait abnormality evaluation is likely to be affected by a clinician's expertise and subjectivity. In the case of the vision-based analysis, the equipment installation is complex and the equipment price is high, so it is possible to operate the vision-based analysis in a hospital, but there are limitations in operating the vision-based analysis outside the hospital with the concept of precision medicine to detect the occurrence of potential diseases in advance or early and link a patient with the hospital at an early stage.

Precision medicine is the concept of personalized, participatory, predictive, and preventive (4P) medicine that expands the space of medical services from hospitals to society and homes, analyzes individual user activities (Personalization) to predict correlation with diseases (Prediction), and prevents or treats early diseases in connection with hospitals when the likelihood of disease occurrence increases, to reduce social burden due to medical costs and the reduced quality of life (QoL).

However, in places (e.g., home, company, etc.), other than the hospital, to which the concept of precision medicine is applied, specialized clinicians cannot reside, and complex and expensive equipment cannot be provided, making it impossible to quantitatively and objectively analyze the gait of an individual (a person). Therefore, it is necessary to overcome the limitations of impossibility of accurate gait analysis and furthermore complement the voluntary and independent participation of a subject through interaction.

A treadmill is an exercise machine that can be easily accessed at home or in a gym, and it is easy to manage and control the intensity of exercise, such as speed and incline, so that the speed of the treadmill can be adjusted to suit the subject.

Furthermore, when the speed of the treadmill is kept constant, there is no significant mechanical difference between walking on the treadmill and walking on the normal ground. Cases where joint angles of the legs during walking also reported similarities in joint motion and kinematics between both types of walking.

Therefore, an aspect of the present disclosure is to propose a technical solution for quantitatively and objectively analyzing gait using an existing treadmill that can be easily accessed in general homes and gyms.

### SUMMARY OF THE INVENTION

The present disclosure has been designed in consideration of the above-described circumstances. An aspect to be achieved in the present disclosure is to implement a specific technical solution for quantitatively and objectively analyzing gait on an existing treadmill by using an adapter sensor that can be attached to an easily accessible motorized treadmill in a simple manner without modification.

Another aspect to be achieved in the present disclosure is to implement a technical solution that can obtain accurate gait characteristics of users even when the type of treadmill, the characteristics of a motor driving the treadmill, and the set speed are changed, and enables accurate interaction using the treadmill in providing gait analysis-based services.

Another aspect to be achieved in the present disclosure is to implement a technical solution that can accurately identify each gait posture phase defined in a specific vision-based reference model through the instantaneous current value of a motor of a treadmill.

A gait analysis device according to one embodiment of the present disclosure may include: a data collector configured to collect sensing data obtained by sensing an instantaneous current value that is supplied to a treadmill to operate a motor of the treadmill; a data generator configured to analyze the sensing data to generate a user's gait data; and a controller configured to send the gait data so that the gait data is displayed, recorded, or transmitted.

The data generator may be configured to set a threshold that is a maximum value of the instantaneous current value for a predetermined time in an idle state, and to generate the gait data by analyzing the sensing data from a time point when the instantaneous current value exceeds the threshold.

The data generator may be configured to reset the threshold in case that a rotation speed of the motor is reset.

The data generator may be configured to extract a first step and a second step of the user by continuous outer envelopes extracted from the sensing data. The first step and the second step may be repeated sequentially.

The data generator may be configured to distinguish a step of the user's left foot from a step of the user's right foot by defining a series of the first steps as index walking in case that maximum values of envelopes of the series of the first steps are greater than values obtained by adding a reference value to maximum values of envelopes of a series of the second steps.

The data generator may be configured to analyze the collected sensing data to generate the gait data, which is divided into gait posture phases (stance phase) defined in a specific vision-based reference model.

The data generator may be configured to extract the user's step by using a continuous outer envelope extracted from the sensing data, and generate the gait data by distinguishing, in the envelope constituting the one step, an LR section in which a current value increases, an MS section in which the current value decreases after the LR section, and a PW section in which the current value decreases and reaches a minimum value after the MS section.

The data generator may be configured to distinguish, in the envelope constituting the one step, a TS section in which the current value decreases after the MS section. In this case, the TS section is before the PW section, and a decreasing slope of the TS section may be less than a decreasing slope of the MS section and a decreasing slope of the PW section.

The data generator may be configured to extract individual steps from continuous outer envelopes extracted from the sensing data, overlap data of the extracted individual steps to generate a heatmap in which data for each step is distributed at each distinction time point within a unit time, and generate the gait data by matching each gait posture phase defined in the specific vision-based reference model to at least one of the heatmap and a median graph derived from the sensing data.

The controller may be configured to use at least one of the heatmap and the median graph as the gait data to: analyze a gait control state based on a distribution degree; or analyze a predicted disease based on at least one among the shape of a curve, a distinction location for each gait posture phase, a slope between the distinction locations, and relative sizes of the distinction locations.

The controller may be configured to calculate the degree of pain and discomfort related to the predicted disease by additionally reflecting the collected sensing data in the analysis in case that the user's predicted disease has been analyzed using the gait data.

The controller may be configured to provide gait-related content to a display device of a user walking on the treadmill, and to control the providing of the gait-related content to the display device based on real-time gait patterns and gait pattern changes analyzed using the gait data.

A gait analysis-based service method according to an embodiment of the present disclosure may include: a data collection operation of collecting sensing data obtained by sensing an instantaneous current value that is supplied to a treadmill to operate a motor of the treadmill; a data generation operation of analyzing the sensing data to generate a user's gait data; and a service providing operation of providing digital content or mileage to the user based on the walking data.

In the data generation operation, a threshold that is a maximum value of the instantaneous current value for a predetermined time in an idle state may be set, and the gait data may be generated by analyzing the sensing data from a time point when the instantaneous current value exceeds the threshold.

In the data generation operation, the threshold may be reset in case that a rotation speed of the motor is reset.

In the data generation operation, a first step and a second step of the user may be extracted by continuous outer envelopes extracted from the sensing data. In this case, the first step and the second step may be repeated sequentially, and a step of the user's left foot may be distinguished from a step of the user's right foot by defining a series of the first steps as index walking in case that maximum values of envelopes of the series of the first steps are greater than values obtained by adding a reference value to maximum values of envelopes of a series of the second steps.

In the service providing operation, a first service may be provided in case that at least one of the first step and the second step meets a first condition.

In the data generation operation, the collected sensing data may be analyzed to generate the gait data, which is divided into gait posture phases (stance phase) defined in a specific vision-based reference model.

In the data generation operation, the user's step may be extracted by using a continuous outer envelope extracted from the sensing data, and the gait data may be generated by distinguishing, in the envelope constituting the one step, an LR section in which a current value increases, an MS section in which the current value decreases after the LR section, and a PW section in which the current value decreases and reaches a minimum value after the MS section.

In the data generation operation, a TS section in which the current value decreases after the MS section may be distinguished in the envelope constituting the one step. In this case, the TS section is before the PW section, and a decreasing slope of the TS section may be less than a decreasing slope of the MS section and a decreasing slope of the PW section.

In the data generation operation, individual steps may be extracted from continuous outer envelopes extracted from the sensing data, overlap data of the extracted individual steps to generate a heatmap in which data for each step is distributed at each distinction time point within a unit time, and the gait data may be generated by matching each gait posture phase defined in the specific vision-based reference model to at least one of the heatmap and a median graph derived from the sensing data.

In the service providing operation, at least one of the heatmap and the median graph as the gait data may be used to analyze a gait control state based on a distribution degree or analyze a predicted disease based on at least one among the shape of a curve, a distinction location for each gait posture phase, a slope between the distinction locations, and relative sizes of the distinction locations.

In the service providing operation, at least one of the heatmap and the median graph as the gait data may be used to analyze at least one among symmetry of both feet, distinction between a dominant foot and a non-dominant foot, gait characteristics, optimal walking speed, optimal running speed, and the amount of energy consumption, based on at least one of the shape of the curve and the area of the curve.

In the service providing operation, gait-related content may be provided to a display device of a user walking on the treadmill, and the providing of the gait-related content to the display device may be controlled based on gait patterns and gait pattern changes analyzed using the gait data.

According to an embodiment of the present disclosure, it is possible to implement a specific technical solution for quantitatively and objectively analyzing gait on an existing treadmill by using an adapter sensor that can be attached to a motorized treadmill in a simple manner without modification.

Accordingly, the present disclosure realizes an environment in which quantitative and objective gait analysis results can be obtained in the form of lifelog data without the need for complex and expensive equipment, thereby enabling the provision of various services (e.g., disease diagnosis services, home training services, content-linked services, and the like) based on gait analysis results, such as enabling disease prediction in the concept of precision medicine.

According to an embodiment of the present disclosure, a threshold that is the maximum value of an instantaneous current value for a predetermined time in an idle state is first set, and gait data is generated by analyzing sensing data from a time point when the instantaneous current value exceeds the threshold. Therefore, accurate gait characteristics of a user may be obtained even when the type of treadmill, the characteristics of a motor driving the treadmill, and the set speed are changed. In addition, accurate interaction using the treadmill may be made in providing gait analysis-based services. In addition, this may be implemented and operated with a relatively low-specification MPU.

According to an embodiment of the present disclosure, the user's step may be extracted by a continuous outer envelope extracted from the sensing data. Gait data may be generated by distinguishing an LR section, an MS section, and a PW section, respectively, in the envelope that constitutes one step. In addition, the gait data may be generated by distinguishing a TS section within the envelope constituting one step. Thus, the instantaneous current value of the treadmill's motor may be used to accurately identify each gait posture phase defined in a specific vision-based reference model.

### BRIEF DESCRIPTION OF THE DRAWINGS

The above and other aspects, features, and advantages of the present disclosure will be more apparent from the following detailed description taken in conjunction with the accompanying drawings, in which:

FIG. 1 illustrates each gait posture phase (stance phase) defined in a vision-based RLA gait phase model.

FIG. 2 is a block diagram illustrating a gait analysis device according to an embodiment of the present disclosure.

FIG. 3 illustrates an environment in which a gait analysis device according to an embodiment of the present disclosure operates.

FIG. 4 illustrates gait data generated according to an embodiment of the present disclosure.

FIG. 5 illustrates an example of sensing data obtained according to an embodiment of the present disclosure.

FIG. 6 illustrates an example of setting a threshold from sensing data obtained according to an embodiment of the present disclosure and obtaining gait data.

FIGS. 7A-7B illustrate sensing data obtained according to an embodiment of the present disclosure, and FIG. 7B illustrates an enlarged view of a section transitioning from an idle section to a walking section in FIG. 7A.

FIGS. 8A-8B illustrate index walking and gait data generated according to an embodiment of the present disclosure.

FIGS. 9A-9B illustrate the concept of normalizing gait data generated according to an embodiment of the present disclosure.

FIGS. 10A to 14 illustrate examples that can be provided by a gait analysis-based disease diagnosis service according to embodiments of the present disclosure.

FIGS. 15 to 17C illustrate examples that can be provided by a gait analysis-based home training service according to embodiments of the present disclosure.

FIGS. 18 to 20 illustrate examples that can be provided by a gait analysis-based content-linked service according to embodiments of the present disclosure.

FIG. 21 illustrates the flow of operations of a gait analysis device according to an embodiment of the present disclosure.

### DETAILED DESCRIPTION OF THE EXEMPLARY EMBODIMENTS

Hereinafter, embodiments of the present disclosure will be described with reference to the accompanying drawings.

The present disclosure relates to a technology for analyzing the gait of a person (a user).

In particular, the present disclosure relates to a technology for quantitatively and objectively analyzing gait on an existing treadmill by using a sensor that can be attached to the treadmill in a simple manner without modification.

Walking, as one of fundamental activities which humans (persons) has been doing for a long time, is used as a popular form of exercise (a fundamental training element) with various physiological benefits. In addition, gait has different characteristics for each person depending on lifestyle, physical characteristics, psychological characteristics, and health conditions, and is thus analyzed and used to estimate the different characteristics.

In particular, kinematic gait stability is known to be correlated with gait speed, muscle strength, and joint range of motion.

That is, many orthopedic or neurological diseases are likely to cause an abnormal gait, and conversely, incorrect gait movements may lead to diseases in the brain, body structures, joints, muscles, and so on. Thus, gait information is recognized as important information that doctors need to consider for medical diagnosis.

There have been cases in which gait information is used to develop algorithms that extract gait features, such as balance, speed, posture, and step length, for patients with specific diseases to determine the severity of the diseases, or is used to track the progress of treatment for patients with degenerative brain diseases.

To achieve this, it is necessary to accurately analyze the gait of the patient (person) to extract and obtain accurate gait features and gait information.

Current gait analysis methods in use include the “Timed Up & Go (TUG)” test, in which a person walks a predetermined distance under an examiner's instructions and the result is evaluated, and vision-based analysis, which uses visual markers and foot pressure sensors.

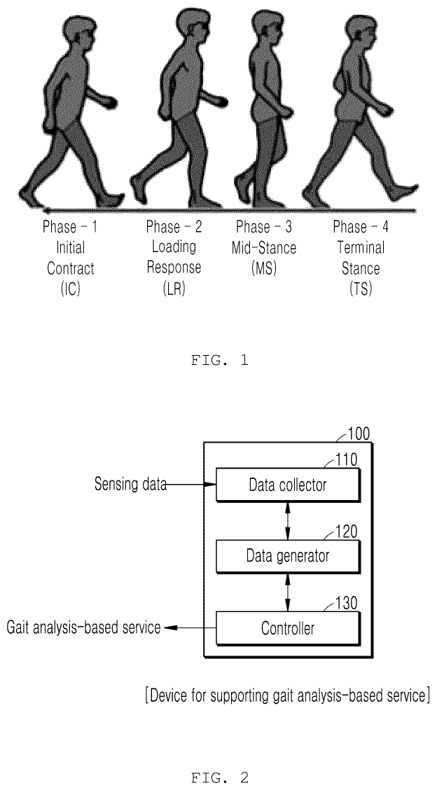

FIG. 1 shows each gait posture phase (stance phase) as defined in the RLA gait phase model established by Dr. Jacquelin Perry at the Rancho Los Amigos Rehabilitation Clinic, among analysis models based on the vision-based analysis.

In general, a gait cycle may be from a time point when the heel of a left or right foot contacts the ground to a time point when the heel of the same foot contacts the ground again to begin a new gait.

Therefore, when a person (user) with a visual marker attached to a designated body part walks, a gait cycle is divided into phases by the movement according to walking. In the RLA gait phase model, gait is divided into two phases (stance phase and swing phase), and is subdivided into eight phases (initial contact (IC), loading response (LR), mid stance (MS), terminal stance (TS), pre-swing (PW), initial swing, mid swing, and terminal swing).

FIG. 1 illustrates four phases (initial contact (IC), loading response (LR), mid stance (MS), and terminal stance (TS) that belong to the stance phase as defined by the RLA gait phase model.

As will be described in detail later, in the present disclosure, gait data is generated for a section (step) in which the foot directly applies a load to a pad of a treadmill (the “pad” of the treadmill described in the embodiment of the present disclosure may be referred to as a “belt”), so that gait data that is distinguished as a stance phase (IC, LR, MS, and TS) is generated.

Referring back to the current gait analysis methods in use, in the case of the “Timed Up & Go (TUG)” test, the accuracy of gait abnormality evaluation is likely to be affected by a clinician's expertise and subjectivity. In the case of the vision-based analysis, the equipment installation is complex and the equipment price is high, so it is possible to operate the vision-based analysis in a hospital, but there are limitations in operating the vision-based analysis outside the hospital with the concept of precision medicine to detect the occurrence of potential diseases in advance or early and link a patient with the hospital at an early stage.

Precision medicine is the concept of personalized, participatory, predictive, and preventive (4P) medicine that expands the space of medical services from hospitals to society and homes, analyzes individual user activities (Personalization) to predict correlation between the activities and diseases (Prediction), and prevents or treats early diseases in connection with hospitals when the likelihood of disease occurrence increases, to reduce social burden due to medical costs and the reduced quality of life (QoL).

However, in places (e.g., home, company, etc.), other than the hospital, to which the concept of precision medicine is applied, specialized clinicians cannot reside and complex and expensive equipment cannot be provided, making it impossible to quantitatively and objectively analyze the gait of an individual (a person). Therefore, it is necessary to overcome the limitations of impossibility of accurate gait analysis, and furthermore, complement the voluntary and independent participation of a subject through interaction.

A treadmill is an exercise machine that can be easily accessed at home or in a gym, and it is easy to manage and control the intensity of exercise, such as speed and incline, so that the speed of the treadmill can be adjusted to suit the subject.

Furthermore, when the speed of the treadmill is kept constant, there is no significant mechanical difference between walking on the treadmill and walking on the normal ground. Cases where joint angles of the legs during walking also reported similarities in joint motion and kinematics between both types of walking.

Therefore, an aspect of the present disclosure is to propose a technical solution for quantitatively and objectively analyzing gait using an existing treadmill that can be easily accessed in general homes and gyms.

More specifically, an aspect of the present disclosure is to implement a specific technical solution for quantitatively and objectively analyzing gait on an existing treadmill by using an adapter sensor that can be attached to a motorized treadmill in a simple manner without modification.

FIG. 2 illustrates the configuration of a device for realizing technical features proposed in the present disclosure, i.e., a gait analysis device.

As illustrated in FIG. 2, according to an embodiment of the present disclosure, a gait analysis device 100 may include a data collector 110, a data generator 120, and a controller 130.

All or at least some of the elements of the gait analysis device 100 may be implemented in the form of hardware modules, in the form of software modules, or in a combination of hardware and software modules.

Here, software modules may be understood as instructions executed by a processor configured to control computation in the gait analysis device 100. The instructions may be loaded into a memory in the gait analysis device 100.

In the end, the gait analysis device 100 according to an embodiment of the present disclosure, through the aforementioned elements, implements a technology proposed by the present disclosure, i.e., a specific technical solution for quantitatively and objectively analyzing gait using an existing treadmill, and hereinafter, each element in the gait analysis device 100 for implementing the technical solution will be described in more detail.

According to an embodiment of the present disclosure, as described later, an instantaneous current value supplied to a treadmill may be sensed by a sensor 20, data (sensing data) of the instantaneous current value sensed by the sensor 20 is transmitted to the gait analysis device 100, and the gait analysis device 100 is configured to analyze the gait of a user based on the sensing data.

In order to operate a motor for operating (rotating) the pad of the treadmill (the “motor” described in the embodiment of the present disclosure refers to a motor for operating (rotating) the pad of the treadmill, unless otherwise specifically limited), information (sensing data) on the instantaneous current value supplied to the treadmill is transmitted to the data collector 110 of the gait analysis device 100.

The data collector 110 collects data (sensing data) of an instantaneous current value for operating the motor of the treadmill. That is, the data collector 110 collects the sensing data when the user walks on the pad, and may collect the sensing data even when the user is not walking on the pad.

In particular, the data collector 110 is responsible for collecting sensing data obtained by sensing instantaneous current values generated by changes in load of the motor of the treadmill accompanying the user's walking.

The magnitude of the current values illustrated in the drawings according to embodiments of the present disclosure may be relative. The unit of the current values illustrated in the drawings according to embodiments of the present disclosure may be mA.

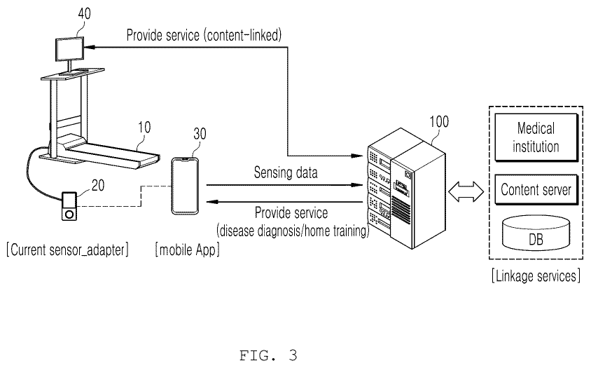

Referring to FIG. 3 illustrating an environment in which a gait analysis device 100 according to the present disclosure operates, the present disclosure is based on a configuration in which a sensor 20 attachable to a treadmill 10 in a simple manner is used.

For example, the sensor 20 may be an adapter-type current sensor, and may sense an instantaneous current value generated in the treadmill 10.

The largest power consumer in the treadmill 10 is a motor that repeatedly rotates a pad (a belt) of the treadmill 10. Thus, the instantaneous current value sensed by the sensor 20 may be considered to be a current signal generated by the motor of the treadmill 10.

That is, the sensor 20 senses an instantaneous current value due to a change in the motor load of the treadmill 10 to monitor a change in the instantaneous current value due to the change in the motor load in real time.

Various elements may be applied to the sensor 20. For example, a shunt register may be applied to precisely sense and monitor AC current changes of 50 Hz or 60 Hz with sufficient sampling period.

In the present disclosure, sensing data sensed by the sensor 20 is transmitted to the gait analysis device 100.

In this case, the sensing data from the sensor 20 may be transmitted directly to the gait analysis device 100, or may be transmitted via a separate device (e.g., a smartphone, etc.).

In one example, the sensing data from the sensor 20 may be transmitted using short-range communication (e.g., Bluetooth) to a mobile app installed on a smartphone 30 and then transmitted by the mobile app to the gait analysis device 100.

Accordingly, when a person (a user) initiates walking on the treadmill 10, the data collector 110 may collect real-time sensing data sensed by the sensors 20 by using the various transmission methods as described above.

The data generator 120 analyzes the collected sensing data to generate gait data. The data generator 120 may generate gait data based on the magnitude of the sensing data at a specific time point, the magnitude of the sensing data in a predetermined time interval, the change of the sensing data over time, and the like, and may identify the gait of the user based on the gait data.

In particular, the data generator 120 is responsible for analyzing the above-collected sensing data to generate gait data that is divided into gait posture phases (stance phase) defined by a specific vision-based reference model.

In the present disclosure, gait data is generated by analyzing sensing data obtained by sensing an instantaneous current value of the treadmill 10. Thus, the gait data is generated in a step cycle from a time point when the heel of a left or right foot contacts the ground to a time point when the toes of the same foot leave the ground.

Accordingly, in the present disclosure, an existing model that distinguishes each gait posture phase (stance phase) for a step may be adopted and used as the specific vision-based reference model.

However, in the following embodiments, the RLA gait phase model will be described as a specific reference model.

That is, in the present disclosure, gait data may be generated for a step including a section where the foot directly applies a load to the pad of the treadmill 10, i.e., from a time point when the heel of the left or right foot contacts the pad of the treadmill 10 to a time point when the toes of the same foot leave the pad, and gait data distinguished as a stance phase (IC, LR, MS, and TS) defined in the RLA gait phase model may be generated.

Furthermore, according to an embodiment of the present disclosure, gait data which is divided into stance phase (IC, LR, MS, TS) and swing phase (PW) defined in the RLA gait phase model may be generated.

More specifically, the data generator 120 may extract individual steps from a continuous outer envelope extracted from the sensing data.

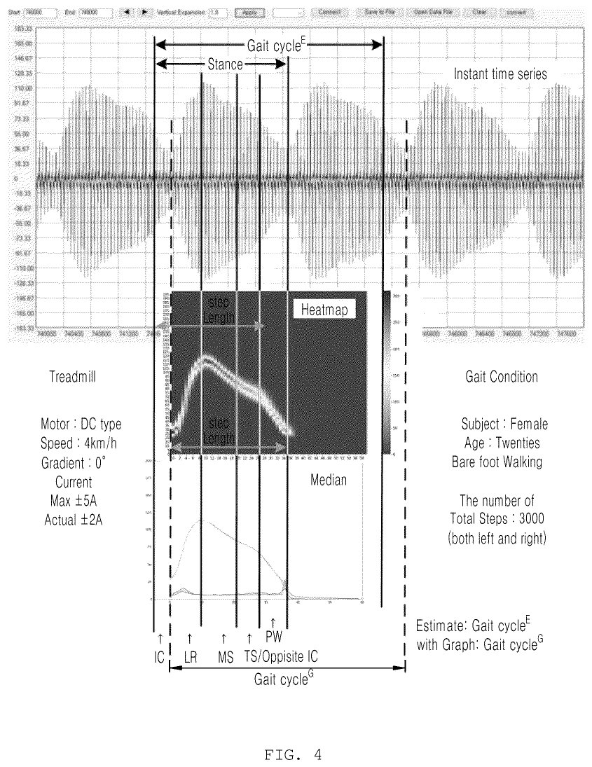

Referring to FIG. 4, it is assumed that sensing data is obtained by walking on a treadmill at a speed of 4 km/h for 30 minutes.

In each of the graphs illustrated in the drawings describing embodiments of the present disclosure, the x-axis (horizontal direction) represents time and the y-axis (vertical direction) represents a current value, unless otherwise specified.

The graph above shows sensing data (changes in instantaneous current value) sensed at intervals of 1/60 seconds, assuming a 60 Hz AC current.

The data generator 120 may extract a continuous outer envelope from the sensing data (changes in instantaneous current value) illustrated at the upper side of FIG. 4, and may automatically extract individual steps from the extracted continuous outer envelope.

In this way, when a typical adult walks at a speed of 4 km/h for 30 minutes, the adult would take approximately 3000 steps, and thus approximately 3000 steps may be extracted.

The data generator 120 may overlap data of the extracted individual steps to generate a heatmap in which data for each step is distributed at each distinction time point within a unit time.

A unit time refers to one second, and a distinction time point within a unit time may be related to a sampling period at which sensing data is sensed (e.g., 1/60 seconds for 60 Hz AC current and 1/50 seconds for 50 Hz AC current).

That is, the data generator 120 may overlap data of individual steps extracted from the sensing data (changes in instantaneous current value) to generate a heatmap in which the distribution of data for each step is visually shown at intervals of 1/60 seconds as approximately 3000 steps overlap (see the middle graph in FIG. 4).

Furthermore, the data generator 120 may derive a median graph from the sensing data (changes in instantaneous current value) (see the bottom graph in FIG. 4)

In one embodiment, the data generator 120 may generate gait data by matching each gait posture phase (e.g., IC, LR, MS, or TS) defined in the above-described specific reference model (e.g., the RLA gait phase model) to at least one of the heatmap and the median graph.

In one embodiment, the data generator 120 may generate gait data by matching each gait posture phase (e.g., IC, LR, MS, TS, or PW) defined by the above-described specific reference model (e.g., the RLA gait phase model) to at least one of the heatmap and the median graph.

FIG. 4 shows a graph of sensing data (changes in instantaneous current value) on the top, a heatmap on the middle, and a median graph on the bottom.

As such, the present disclosure realizes features wherein changes in load of the motor of the treadmill 10 depending on the gait of a person (a user), i.e., instantaneous current values, are sensed by using the sensor 20 attachable to the treadmill 10 in a simple manner without modification; and gait data (a heatmap and a median graph) to which each gait posture phase (e.g., IC, LR, MS, TS, or PW) defined by the RLA gait phase model is matched is generated from the sensing data.

As can be seen in FIG. 4, in the gait data (the heatmap and the median graph) generated in the present disclosure, the instantaneous current value increases when the gait applies a load to the motor of the treadmill 10. Therefore, when the gait progresses from an IC phase, where the heel contacts the pad of the treadmill 10, to an LR phase, the heel presses the pad, and thus the instantaneous current value rapidly increases, and reaches a maximum value in the LR phase.

Subsequently, in an MS phase, the instantaneous current value decreases gradually because the foot becomes adapts to the rotation of the pad due to the characteristics of the treadmill 10 and only the load due to body weight remains, and in a TS phase as the last phase, the toe strongly pushes off the pad, causing the last load to disappear rapidly.

Among the generated gait data, the heatmap may be applied to various visual gait analysis technologies such as convolution neural network (CNN), and the median graph may be applied to various numerical and quantitative gait analysis technologies. Therefore, the heatmap and the median graph may be used complementarily.

As such, in the present disclosure, the sensor 20 attachable to the treadmill 10 in a simple manner may be used to generate gait data (the heatmap and the median graph) for quantitative and objective analysis of gait using the existing treadmill 10, thereby realizing an environment in which quantitative/objective gait analysis results can be obtained in the form of lifelog data without the need for complex and expensive equipment such as a visual marker and a foot pressure sensor. In addition, in the present disclosure, it is possible to measure dynamic changes in time intervals that may occur during walking for a long time, beyond the limitations of measurement in limited time and space using the existing method.

Meanwhile, various types of treadmills widely available and in use may have different types of rated power and different input voltages, resulting in differences in the amount of variation in current values for the same load, requiring normalization to make the values constant is required for analysis on the same criteria.

Accordingly, in the present disclosure, for the gait data (the heatmap and the median graph) generated as described above, a normalization process for analysis of the same criteria may be performed.

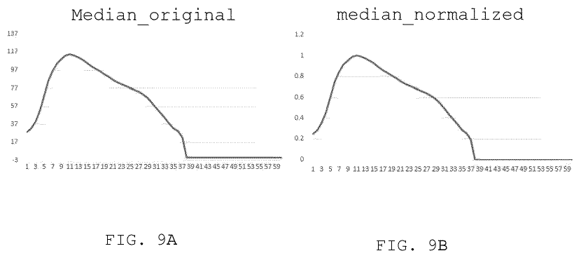

For example, FIGS. 9A and 9B illustrates the original median graph on the left and the normalized result of the median graph on the right.

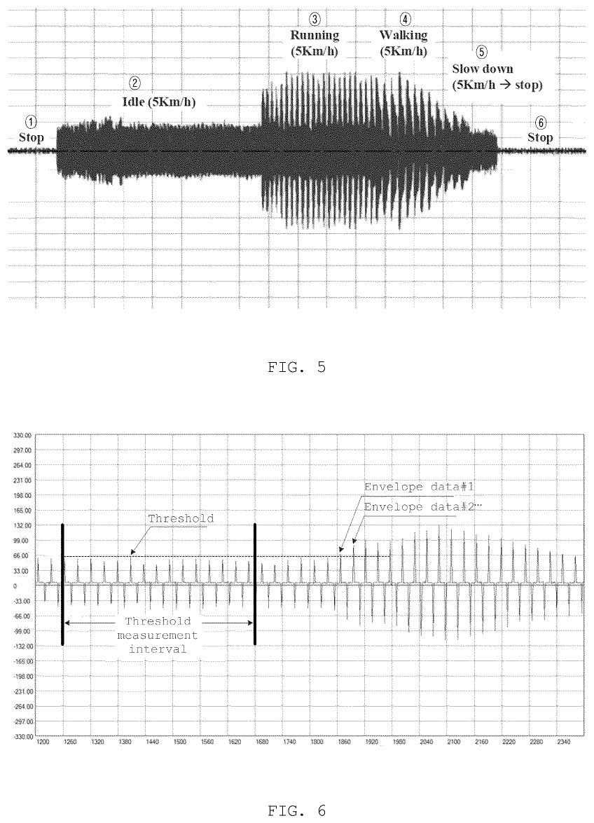

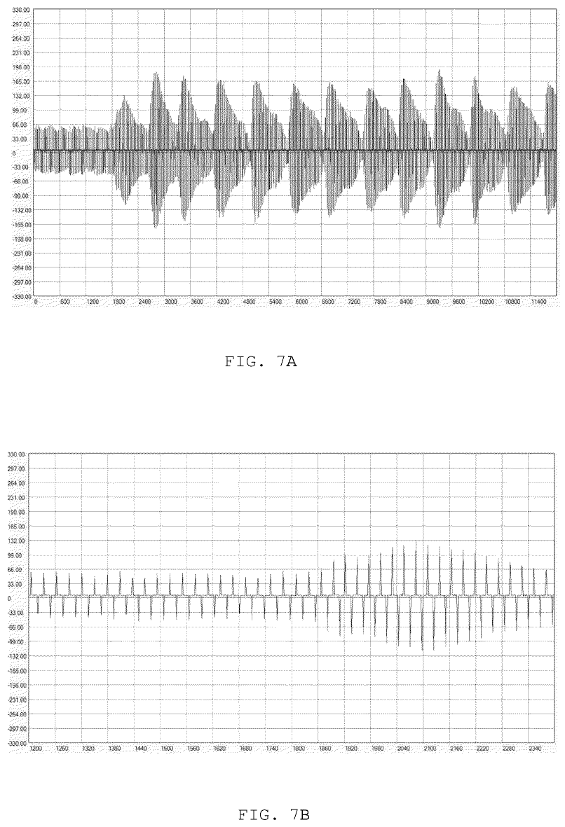

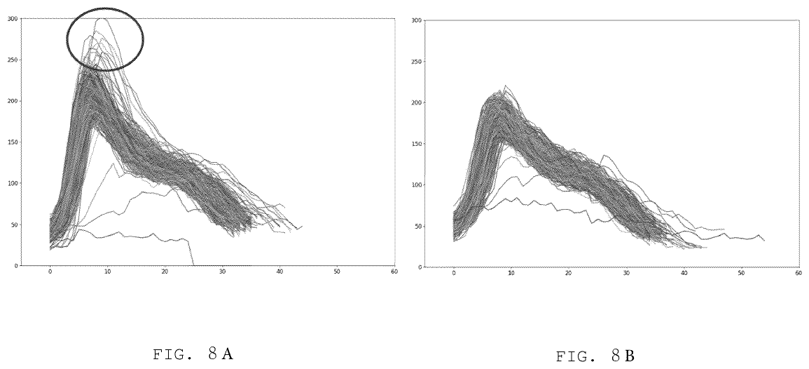

FIG. 5 illustrates sensing data obtained according to an embodiment of the present disclosure. FIG. 6 illustrates an example of setting a threshold value from sensing data obtained according to an embodiment of the present disclosure and obtaining gait data. FIGS. 7A and 7B illustrates sensing data obtained according to an embodiment of the present disclosure. FIG. 7B illustrates an enlarged view of a section transitioning from an idle section to a walking section in FIG. 7A.

In an embodiment of the present disclosure, the data generator 120 collects sensing data obtained by sensing instantaneous current values in an idle state and a use state. The idle state refers to a state in which a pad of a treadmill is rotating while a user is not on the pad of the treadmill. The use state refers to a state in which the pad of the treadmill is rotating while the user walks or runs on the pad of the treadmill.

The maximum value of the sensing data (instantaneous current waveform) in the idle state is less than the maximum value of the sensing data (instantaneous current waveform) in the use state. The average value of the instantaneous sensing data in the idle state for a predetermined time is less than the average value of the sensing data in the use state.

In FIG. 5, 1) is a section in which there is no rotation of the pad when the treadmill is powered on, (2) is a section in the idle state, 3) is a section in which a user is running during the use state, 4) is a section in which the user is walking during the use state, 5) is a section in which the rotation speed of the pad gradually decreases, and 6 is a section in which the rotation of the pad stops.

As illustrated in FIG. 6, in an embodiment of the present disclosure, the data generator 120 may set a threshold, which is a maximum value of the instantaneous current value for a predetermined time (a time corresponding to a threshold measurement interval) in the idle state. The predetermined time may be from tens of milliseconds to several seconds.

In an embodiment of the present disclosure, the data generator 120 may be configured to generate gait data by analyzing the sensing data from a time point at which the instantaneous current value exceeds the threshold. Accordingly, the gait analysis device 100 may recognize the sensing data after the threshold value is exceeded as data related to the gait of the user, and may measure the maximum value of the cycle repeated at a specific frequency every second as an envelope and transmit the maximum value.

The data generator 120 may be configured to reset the threshold when the rotation speed of a motor rotating the pad of the treadmill is reset.

Accordingly, accurate gait characteristics of the user may be obtained even when the type of treadmill, the characteristics of the motor driving the treadmill, and the set speed are changed. In addition, accurate interaction using the treadmill may take place in providing services based on gait analysis. Furthermore, the gait analysis device 100 may be implemented and operated even with a relatively low-specification microprocessor unit (MPU).

FIGS. 8A and 8B illustrates index walking and gait data generated according to an embodiment of the present disclosure. FIG. 8A shows overlapping median graphs depicting the gait of a user's left foot, and FIG. 8B shows overlapping median graphs depicting the gait of the user's right foot.

As illustrated in FIGS. 9A and 9B, in the case of a median graph, normalization may be performed by calculating a denominator that takes the maximum value of the data in the original median graph as 1, and dividing each value in the median graph by the calculated value to give the maximum value with a margin of 1+0.5.

Furthermore, although not illustrated in FIGS. 9A or 9B, normalization may be performed by calculating a denominator that takes the maximum value of the data in the original median graph as 1, and dividing each value in the heatmap by the calculated value to give the maximum value with a margin of 1+0.5.

In this way, the present disclosure may consistently generate gait data (the heatmap and the median graph), which can be used to analyze gait using the treadmill 10, regardless of the type of treadmill.

On the other hand, in the present disclosure, gait data (the heatmap and the median graph) is generated from sensing data obtained by sensing changes in load of a motor of the treadmill 10, i.e., instantaneous current values, so that it should be possible to distinguish between the left and right feet based on only the sensing data without a separate identifier (annotation).

Accordingly, the present disclosure proposes index walking, in which the left and right feet can be distinguished by using sensing data alone, based on the fact that when walking, the feet alternately contact the pad of the treadmill 10, and the alternation order of the feet (e.g., left foot-right foot-left foot-right foot . . . ) does not change unless the feet are switched.

In an embodiment of the present disclosure, the data generator 120 extracts a first step and a second step of a user by a continuous outer envelope extracted from the sensing data. At this time, the first step and the second step are repeated sequentially. When the first step is the step of the left foot, the second step is the step of the right foot. If the first step is the step of the right foot, the second step is the step of the left foot.

When maximum values of envelopes of a series of first steps are greater than values obtained by adding a reference value (RV) to maximum values of the envelope of a series of second steps, the series of first steps are defined as index walking, thereby distinguishing steps of the user's left foot from steps of the user's right foot.

Specifically, index walking is a method in which in the initial gait, one (e.g., the left) foot is walked strongly and the left foot is distinguished from sensing data, thereby distinguish between the left foot and the right foot in subsequent gaits.

When the series of first steps and the series of second steps are repeated, the first steps may be defined as index walking if maximum values of envelopes of the first steps are A1 to An, maximum values of envelopes of the second steps are B1 to Bn, and A1 to An are greater than values obtained by adding a reference value (RV) to B1 to Bn, respectively (e.g., A1>B1+RV, A2>B2+RV, A3>B3+RV, . . . An>Bn+RV).

To this end, in the present disclosure, a user walking on the treadmill 10 is instructed to take a stronger step with one foot than with the other foot during the initial gait.

In this case, the collected sensing data may include a specific pattern of index walking section in which the user takes a stronger step with one foot than with the other foot. Here, the degree of strength may refer to a degree greater than the reference value (RV).

Accordingly, the data generator 120 may generate gait data by distinguishing, based on the index walking section present in the collected sensing data, the one foot, which takes a stronger step, as either the left or right foot predefined for index walking, and distinguishing the other foot as the remaining right or left foot.

That is, when it is defined that the left foot takes a stronger step for index walking, the data generator 120 may generate gait data by distinguishing, based on the index walking section present in the collected sensing data, one foot taking a stronger step as the predefined left foot for index walking and distinguishing the other foot as the remaining right foot.

In FIG. 8A, a red circle indicates a step by index walking, and accordingly, it is possible to accurately distinguish between a step by the left foot from a step by the right foot.

In order to guide a user to perform index walking, the gait analysis device 100 may be configured to control a display, a speaker, or the like to expose index walking guidance information to the user.

In one example, the gait analysis device 100 may transmit guidance information (such as audio information or visual information) such as “Please step on the pad strongly with the left foot and lightly with the right foot for the initial about 10 seconds” to the user through the speaker or the display, and the user may walk by stepping on the pad strongly with the left foot and lightly with the right foot for about 10 seconds after receiving the guidance information.

As described above, in the present disclosure, the sensor 20 attachable to the treadmill 10 in a simple manner may be used to generate gait data (the heatmap and the median graph) for quantitatively and objectively analyzing a gait using the existing treadmill 10, thereby realizing an environment in which quantitative and objective gait analysis results of an individual can be obtained in the form of lifelog data without the need for complex and expensive equipment.

The controller 130 may process the gait data, store the gait data, and transmit the gait data externally.

The controller 130 may transmit the gait data to the display or the speaker such that the gait data may be visually displayed on the display or transmitted to the user via the speaker.

The controller 130 may store the gait data in a storage medium inside the gait analysis device 100, and may transmit the gait data to a device outside the gait analysis device 100.

The controller 130 may be responsible for providing a gait analysis-based service to the user by using the gait data generated by the data generator 120.

That is, as illustrated in FIG. 3, the controller 130, based on linkage with various linkage services (e.g., medical institutions, content servers, various DBs, etc.) outside the gait analysis device 100, may provide various gait analysis-based services by using the gait data (the heatmap and the median graph) generated to analyze the gait using the existing treadmill 10.

Hereinafter, descriptions will be made of scenarios for various gait analysis-based services that can be provided using gait data (the heatmap and the median graph) generated by the present disclosure.

First, the controller 130 may cause the gait data generated by the data generator 120 to be used for gait analysis in a specific existing reference model.

In this regard, according to one embodiment, the controller 130 may derive and provide parameters required by the specific reference model from gait data generated by the data generator 120 to enable the gait of the user to be analyzed without using a visual marker in the specific reference model.

Earlier, the RLA gait phase model was mentioned as a specific reference model. Therefore, continuing to assume the RLA gait phase model, the controller 130 may derive parameters required by the RLA gait phase model from the gait data (the heatmap and the median graph) generated by the data generator 120.

For example, in the RLA gait phase model, when a person (a user) wearing a visual marker walks, a gait cycle is divided into phases based on movement according to the walking, a step length and a stride length are specified, and gait analysis is performed using the specified values.

Here, the step length is the distance from a point where the heel of either the left or right foot contacts the ground to a point where the heel of the other foot contacts the ground, and the stride length is the distance from a point where the heel of either the left or right foot contacts the ground to a point where the heel of the same foot contacts the ground.

In the present disclosure, the parameters required by the RLA gait phase model, i.e., step length and stride length, may be calculated and derived from the gait data (the heatmap and the median graph) generated by the data generator 120.

Specifically, in the case of step length, if the time required for a single step is 0.57 seconds while walking at a speed of 4 km/h as illustrated in FIG. 4, the step length may be derived by calculating Equation 1 below.

$$( \frac{4}{3 , 600} ( km / s ) \times 0.57 ( s ) ) \times 1 , 000 = 0.63 ( m ) \qquad [ Equation 1 ]$$

On the other hand, in the case of stride length, as illustrated in FIG. 4, when walking is performed at a speed of 4 km/h and sensing data is distributed from point 0 to point 35 along the x-axis (0 to 60) on the gait data (the heatmap and the median graph) generated through the above-described process, the stride length may be derived by calculating Equation 2 below.

$$\to x - axis 35 \to ( 35 / 60 ) sec = 0.583 sec \times 2 = 1.167 sec \to 1.297 \qquad [ Equation 2 ]$$

$$( \frac{4}{3 , 600} ( km / s ) \times 1.167 ( s ) ) \times 1 , 000 = 1.297 ( m )$$

Equation 2 is used for finding the stride length when the step length of the left foot is equal to the step length of the right foot. When the step length of the left foot is different from the step length of the right foot due to the user's walking habits and medical conditions, the stride length may be derived by adding the results of Equation 1 for the feet instead of Equation 2.

In this way, necessary parameters, i.e., step length and stride length, may be obtained for gait analysis in the RLA gait phase model. Therefore, it will be possible to analyze the gait of the user without using a visual marker.

Thus, the present disclosure has the effect of combining with existing gait analysis technologies such as the RLA gait phase model based on the realization of an environment in which quantitative/objective gait analysis results of individuals can be obtained in the form of lifelog data by using the existing treadmill 10 without complex and expensive equipment.

According to another embodiment, the controller 130 may provide a gait analysis-based disease diagnosis service by using at least one of a heatmap and a median graph as gait data generated by the data generator 120.

In this regard, the controller 130 may use at least one of the heatmap and the median graph to provide a gait analysis-based disease diagnosis service which analyzes a gait control state based on a distribution degree or analyzes a predicted disease based on at least one among the shape of a curve, a distinction location for each gait posture phase, a slope between the distinction locations, and the relative size of the distinction locations.

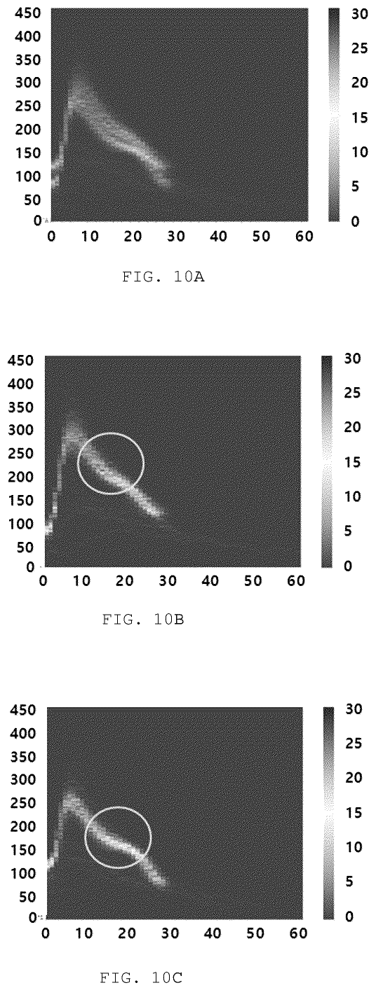

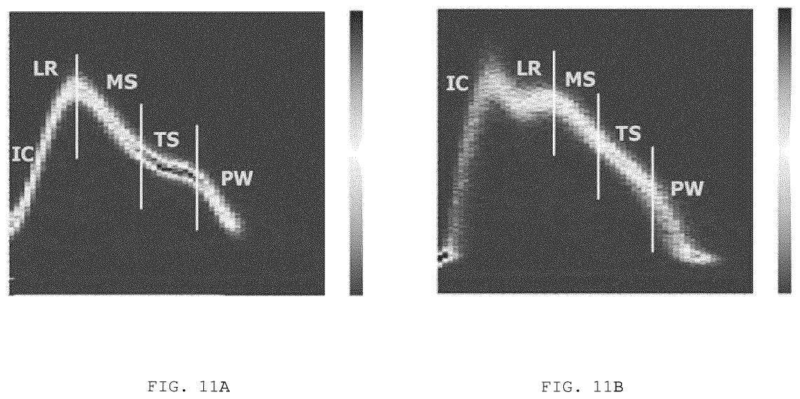

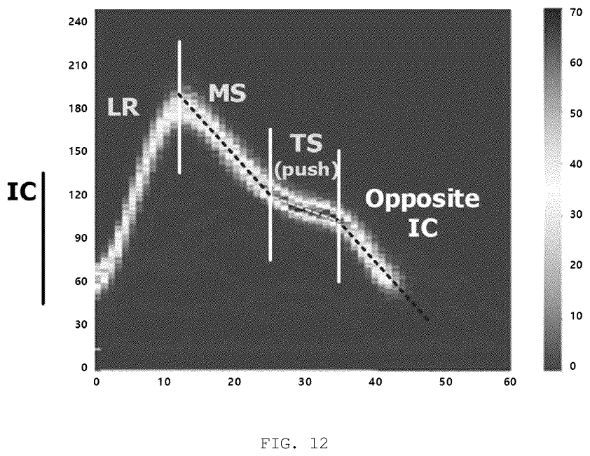

Hereinafter, examples that may be provided by a gait analysis-based disease diagnosis service according to embodiments of the present disclosure will be described with reference to FIGS. 10 to 14.

FIGS. 10A-10C illustrates gait data (heatmaps) generated by collecting and analyzing sensing data in an index walking method according to the present disclosure. FIG. 10A illustrates gait data of both feet, FIG. 10B illustrates gait data of the left foot, and FIG. 10C illustrates gait data of the right foot.

As can be seen in FIG. 10A, the degree of matching between both feet is not high. In particular, it can be seen that in FIG. 10C, the MS phase (portion inside a circle) of right foot has a high repetition characteristic, resulting in good joint control, while in FIG. 10B, the MS phase (portion inside a circle) of the left foot has a low repetition characteristic, resulting in poor joint control.

This situation is more likely to occur when the left side is uncontrolled and more force is applied to the left side, and may be occur when the joints in the left foot of a patient with a left femur fracture are not properly controlled during gait.

Accordingly, the controller 130 may analyze the gait control state and the predicted disease based on a distribution degree and the shape of a curve from the gait data (the heatmap) of FIG. 10, and may analyze the left femur fracture as the predicted disease.

FIGS. 11A and 11B illustrates gait data (heatmaps) generated by collecting and analyzing sensing data according to the present disclosure.

FIG. 11A on the left illustrates gait data (a heatmap) generated for the gait of a healthy person according to the present disclosure, and FIG. 11B on the right illustrates gait data (a heatmap) generated for the gait of a person predicted to have a knee joint disease (e.g., knee fusion).

In the gait data (the heatmap) illustrated in FIG. 11A on the left, it may be found that in IC and MS phases, the heatmap is colored red, with high repetition matching. This signifies that in the above phases, the foot does not consciously apply a strong load or a particular load to the pad of the treadmill, each step has a high degree of matching, and gait control is well executed.

Accordingly, the controller 130 may analyze the gait control state based on the distribution degree and the shape of a curve from the gait data (the heatmap) illustrated in FIG. 11A on the left, and may analyze that the gait control is excellent.

On the other hand, in the gait data (the heatmap, right) illustrated in FIG. 11B on the right, it can be seen that after the IC phase, the center of gravity of the body shifts very rapidly due to poor flexion movement of the knee and is not stabilized, and in the MS phase, significant load is applied to the knees and foot (the area of the bottom part of a heatmap curve increases), and the degree of distribution of each gait posture phase is increasing.

Accordingly, the controller 130 may analyze a predicted disease based on the shape of a curve, a distinction location for each gait posture phase, and the slope between the distinction locations from the gait data (the heatmap) illustrated in FIG. 11B on the right, and may analyze the predicted disease as a knee joint disease (e.g., knee fusion).

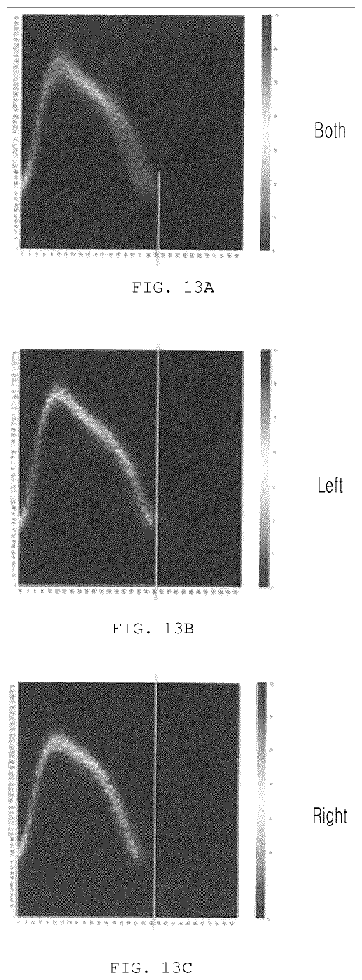

FIG. 12 illustrates one heatmap graph according to an embodiment of the present disclosure.

As described above, in an embodiment of the present disclosure, the data generator 120 may be configured to analyze collected sensing data to generate gait data that is divided into gait posture phases (stance phase) defined in a specific vision-based reference model.

Specifically, the data generator 120 extracts a user's steps by continuous outer envelopes extracted from sensing data. As illustrated in FIG. 12, a heatmap graph may be obtained by overlapping the envelopes.

Gait data may be generated by distinguishing, in the envelope of one step, a loading response (LR) section, where a current value increases, a mid-stance (MS) section, where the current value decreases after the LR section, and a pre-swing (PW) section, where the current value decreases after the MS section but reaches a minimum, respectively (see FIGS. 11A and 11B). In FIG. 12, the PW section is indicated as an Opposite IC section (initial contact section of the opposite foot).

The IC section may be distinguished as a section preceding the LR section.

The PW section corresponds to the pre-swing section of one foot, and this section may overlap, in whole or in part, with the IC section of the other foot.

The data generator 120 may distinguish, in an envelope constituting one step, a terminal stance section (TS section) in which the current value decreases after the MS section. Here, the TS section is before the PW section, and the decreasing slope of the TS section may be less than the decreasing slope of the MS section and the decreasing slope of the PW section. The slope of each section may be obtained through the ratio of a change in time to a change in current value.

Thus, according to an embodiment of the present disclosure, gait data of one foot may be generated while being divided into an IC section, an LR section, an MS section, a TS section, and a PW section (an opposite IC section), and the gait of the user may be accurately diagnosed by accurately recognizing each step of the gait. Accordingly, when providing a service based on gait analysis, the user can input information very accurately by using a treadmill, and interaction using the treadmill may be very accurately performed.

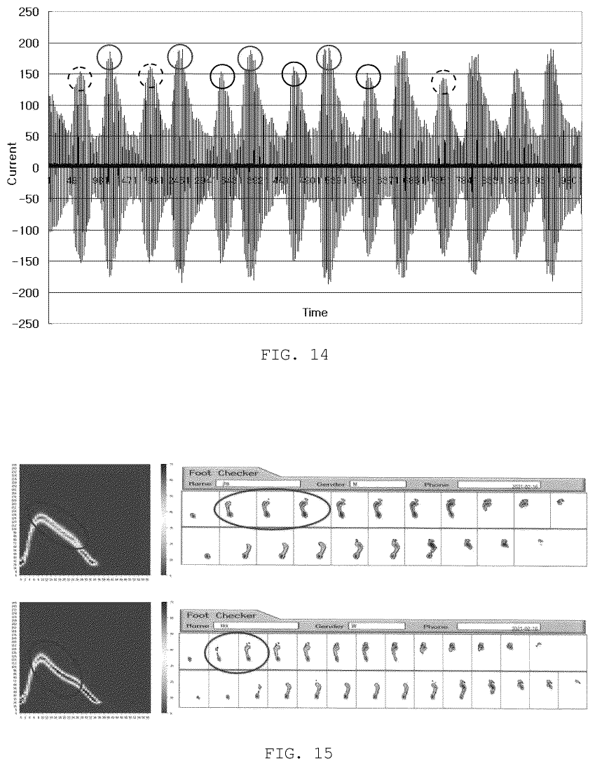

FIGS. 13A-13C illustrates gait data (heatmaps) generated by collecting and analyzing sensing data by means of index walking according to the present disclosure.

As can be seen in FIG. 13A, the degree of matching between both feet is not high. Particularly, it can be seen that the step length of the right foot in FIG. 13C is shorter than that of the left foot in FIG. 13B, and the heatmap distribution is more spread out, and this situation is likely to occur when the right toes are relatively less used in the TS phase.

Accordingly, the controller 130 may analyze a predicted disease based on the distribution degree, the shape of a curve, a distinction location for each gait posture phase, the slope between the distinction locations, and the relative size of the distinction locations from the gait data in FIGS. 13A-13C, and may analyze Achilles tendonitis or plantar fasciitis as the predicted disease.

Furthermore, when the user's predicted disease is analyzed using the gait data (the heatmap or the median graph) as described above, the controller 130 may further reflect the collected sensing data in the analysis to calculate the degree of pain and discomfort due to the predicted disease.

For example, assuming that a femur fracture is analyzed as a predicted disease, the degree of matching between both feet in gait data (a heatmap) is not high as illustrated in FIGS. 10A-10C, and the sensing data analyzed to generate the gait data (the heatmap) shows that data of both feet is jagged as illustrated in FIG. 14.

That is, as can be seen in FIG. 14, one foot (e.g., the right foot) is performing normal gaits (red circles) and the other foot (e.g., the left foot) is performing abnormal gaits (black circles, consciously/unconsciously), and among the abnormal gaits (black circles), solid circles represent patterns of walking and dashed circles represent patterns of running, with running being more frequent.

This implies that, due to pain, the foot (e.g., the left foot) performing the abnormal gaits does not strike the pad vigorously, and instead strikes the pad lightly, as in running, so the frequency of the running patterns (the number of occurrences of dashed circles) among the abnormal gaits (black circles) may be considered to be a pain level.

In light of this, when the user's predicted disease (e.g., femur fracture) is analyzed using the gait data (the heatmap or the median graph), the controller 130 may further reflect the sensing data in the analysis to calculate the frequency of the running patterns (the number of occurrences of dashed circle) among the abnormal gaits (black circle), thereby calculating even the pain level of the predicted disease (e.g., femur fracture).

As described above, the present disclosure may provide a gait analysis-based disease diagnosis service in which predicted diseases are analyzed based on gait data generated for walking using the existing treadmill 10. In addition, in the present disclosure, without being limited to the above-described types of predicted diseases, various kinds of diseases may be predicted by using various gait data analysis technologies (e.g., CNN-Convolutional Neural Network, Ensemble model, etc.), based on linkage with external linkage services (e.g., medical institutions, various DBs, etc.).

In the foregoing, a heatmap as gait data has been described with reference to FIGS. 10 to 13, but this is only for convenience of description. Therefore, it is obvious that the present disclosure may use a median graph as gait data or use both a heatmap and a median graph as gait data, to analyze a predicted disease and provide a gait analysis-based disease diagnosis service.

Accordingly, the present disclosure may provide an analysis-based disease diagnosis service by transmitting information about a predicted disease to a mobile app installed in the smartphone 30 in real time, periodically, or at a set event time point, and may also provide an analysis-based disease diagnosis service by transmitting information about a predicted disease to a related medical institution for use in monitoring long-term disease conditions at home in the field of rehabilitation medicine.

According to another embodiment, the controller 130 may provide a gait analysis-based home training service associated with various types of exercise content by using at least one of a heatmap and a median graph as gait data generated by the data generator 120.

In this regard, the controller 130 may provide a gait analysis-based home training service that uses at least one of a heatmap and a median graph to analyze at least one among symmetry of both feet, dominant/non-dominant foot distinction, gait characteristics, optimal walking/running speed, and the amount of energy consumed, based on at least one of the shape of a curve and the area of the curve.

Hereinafter, examples that may be provided by a gait analysis-based home training service according to embodiments of the present disclosure will be described with reference to FIGS. 15 to 17C.

FIG. 15 illustrates gait data (heatmaps) generated by collecting and analyzing sensing data according to the present disclosure.

The upper and lower parts of FIG. 15 represent gait data from different persons.

Looking at gait data (heatmaps) in the upper and lower parts of FIG. 15, it can be seen that, in the MS phase, the person corresponding to the lower part, compared to the person corresponding to the upper part, experiences a lighter weight shift when the heel of the foot makes contact with the pad, the bottom of the foot contacts the pad, and then the toes of the foot contact the pad. This matches the measurement results using foot pressure sensors shown on the right sides of the gait data (heatmaps).

Accordingly, the controller 130 may analyze gait characteristics based on the shape of curves from the gait data (heatmaps) in the upper and lower part of FIG. 15, and the analysis result may have the same reliability level as the measurement result using an existing foot pressure sensor.

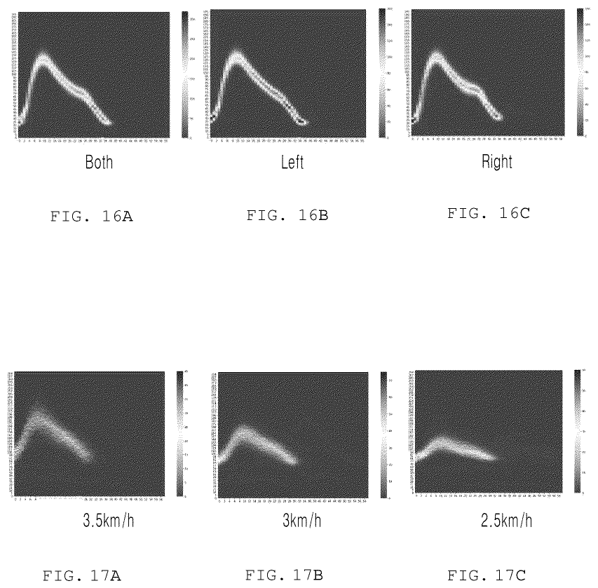

FIGS. 16A-16C illustrates gait data (heatmaps) generated by collecting and analyzing sensing data by means of an index walking according to the present disclosure.

In FIG. 16A, it can be seen that the degree of matching between both feet is high. In FIG. 16C, it can be seen that excessive force is applied to the front part of the right foot when transitioning from an MS phase to a TS phase of the right foot. In FIG. 16B, in can be seen that the left foot is lifted off without a significant change in force compared to the right foot. It may be determined that the symmetry of both feet is high, and that the right foot is the dominant foot and the left foot is the non-dominant foot.

Accordingly, the controller 130 may analyze the symmetry of both feet and the dominant/non-dominant foot distinction based on the shape of curves from the gait data (heatmaps) in FIGS. 16A-16C. In this case, it may be analyzed that the symmetry of both feet is high and the right foot is the dominant foot.

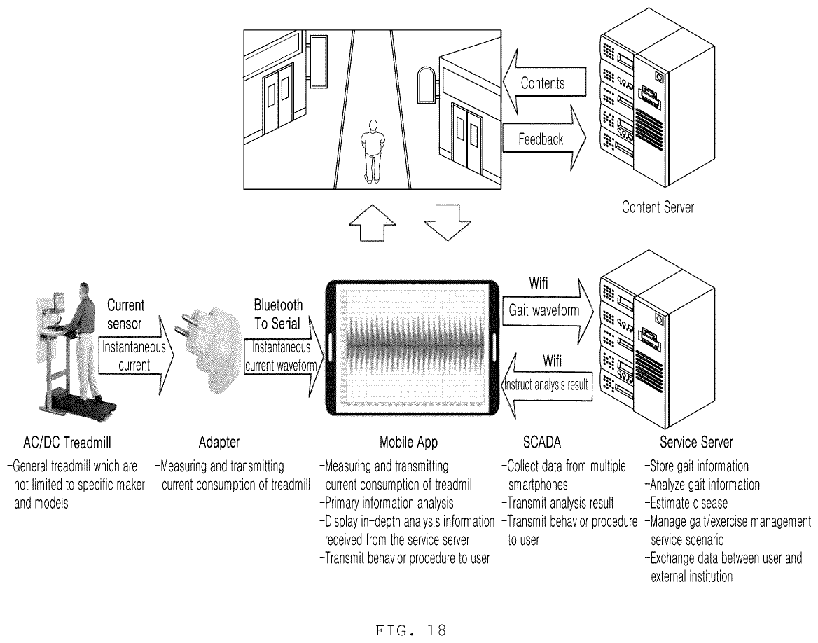

FIGS. 17A-17C illustrate gait data (heatmaps) generated by collecting and analyzing sensing data according to the present disclosure.

FIGS. 17A-17C shows cases in which the same person walks on a treadmill at different speeds. FIGS. 17A, 17B, and 17C show gait data (heatmaps) for speeds of 3.5 km/h, 3 km/h, and 2.5 km/h, respectively.

As illustrated in FIG. 17A, in the case of a speed of 3.5 km/h, it can be seen that the distribution of the heatmap along the y-axis is blurred, indicating low consistency in gait, and that the degree of distribution decreases as the speed decreases.

In particular, in the case of a speed of 2.5 km/h, it can be seen that an LR phase (the leftmost part of the graph), where the foot is stepped on and the load of the first phase of the gait is applied, shows a high repetition characteristic in red, and thus the gait is stable.

The controller 130 may analyze the optimal walking/running speed based on the shape of curves from the gait data (heatmaps) in FIG. 17C. In this case, 2.5 km/h may be analyzed as the optimal walking speed.

Furthermore, the area under each of the heatmap curves of the gait data (heatmaps) may each be considered to be the amount of current consumed, so a larger area may indicate greater energy consumption through walking.

In this regard, the controller 130 may analyze the amount of energy consumed, based on the area of the curve from the gait data (heatmaps).

As described above, in the present disclosure, a gait analysis-based home training service may be provided based on gait data generated for walking using the existing treadmill 10, and the gait data generated while providing the home training service may be transmitted to linkage services (e.g., medical institutions, various DBs, etc.) to be used for various gait analysis technologies (e.g., CNN, ensemble model, etc.).

In the foregoing, a heatmap as gait data has been described with reference to FIGS. 15 to 17, but this is only for convenience of description. Therefore, it is obvious that the present disclosure may also provide a gait analysis-based disease home training service by using a median graph as gait data or using both a heatmap and a median graph as gait data.

Accordingly, in the present disclosure, an analysis-based home training service may be provided by transmitting information about gait analysis to a mobile app installed in the smartphone 30 in real time, periodically, or at a set event time point. In addition, information on gait analysis may be transmitted to a related medical institution for use in monitoring long-term disease conditions at home in the field of rehabilitation medicine, so that the information on gait analysis may also be used in an analysis-based disease diagnosis service.

According to another embodiment, the controller 130 may provide a gait analysis-based content-linked service based on real-time gait patterns and changes in gait patterns that are analyzed using gait data generated by the data generator 120.

In this regard, the controller 130 may provide gait-related content to the display device 40 for a user walking on the treadmill 10.

For example, when a person (a user) initiates gait on the treadmill 10, the controller 130 may provide selected or automatically determined gait-related content to the user's registered display device 40 in conjunction with a content server among linkage services.

The gait-related content may be content including artworks exhibited in corridors of an exhibition hall and in a gallery room of each corridor, descriptions of each artwork, and the like, may be content for exercising together, may be content for traveling to various cities abroad, and may be various other types of content in various fields, which can be incorporated with the treadmill 10, such as walking or running game content.

FIG. 3 illustrates a separate device as the display device 40 on which gait-related content is provided. However, this is only one embodiment, and the smartphone 30 for transmitting sensing data or an augmented reality or virtual reality display device may also be the display device on which gait-related content is provided.

FIG. 18 illustrates an example in which game content with a person walking on the treadmill 10 may be displayed on a screen of the display device 40.

As illustrated in FIG. 18, game content displaying an avatar of a person walking on the treadmill 10 may be displayed on a screen of the display device 40 (the upper part of FIG. 18), and the controller 130 may provide a gait analysis-based content-linked service that controls the provision of gait-related content to the display device 40 by using gait patterns and changes in the gait patterns, analyzed using gait data generated by analyzing sensing data (instantaneous current waveforms) collected from the treadmill 10, as control inputs (the lower part of FIG. 18).

Here, the gait patterns may be analyzed as walking, running, jumping, walking with a bias to one side, etc.

For example, while providing, on the display device (40) for a user walking on the treadmill 10, content in which the user is walking along a corridor in an exhibition hall, when the gait pattern changes from walking to a light jog, content in which the user moved to another corridor or another gallery room may be provided, or when the gait pattern changes from walking to jumping, the content showing a description of an artwork in a gallery room currently being shown may be provided. In this way, a change in gait pattern may be specified and used as a control input for content.

Accordingly, in an example of a scenario in which the controller 130 provides specific content by providing a gait analysis-based content-linked service, artworks are displayed in a corridor as the door opens at the entrance, and when a user starts walking and moves along the corridor to approach an artwork, the artwork may be popped up from the wall toward the user. Then, when the user runs or jumps lightly, the artwork moves with the user (the ambient environment is converted to a simple background without the artwork) and a description of the artwork may be given (using speech (TTS recording), subtitles, or graphics). When the user runs or jumps lightly during the description, the corresponding content may be deselected and a corridor may reappear, so that the user can move to a different description context through a different path (a left or right branch) during the description, and when the user walks with a bias to one side, the user can move to the branch.

In addition, in the present disclosure, a gait pattern and a change in the gait pattern may be used as a next control input.

To control movement within the content, the left or right foot may be changed from walking to running or from running to walking to provide a control input.

A control input may be provided by lightly running with both feet.

A control input may be adjusting the cadence (steps per unit time) of walking or running.

A control input may be provided by adjusting the intensity of waling or running.

A control input may be provided by combining walking or running with the left or right foot.

A control input to determine the direction of travelling may be provided by stomping strongly with the left or right foot.

A control input to determine the direction of travelling may be provided by pressing long with the left or right foot.

Gait (walking, running, or limping) may be used to control a control input for interaction with an avatar and contents represented in virtual reality.

According to another embodiment, as briefly mentioned above, an avatar reflecting a gait motion based on gait data, i.e., an avatar of a person walking on the treadmill 10 may be displayed in gait-related content.

Accordingly, the controller 130 may control the display type of the avatar displayed within the gait-related content based on gait analysis using gait data.

For example, a user may be represented by an avatar such that the user easily recognizes a current gait state and changes therein by using gait data, and the display type of the avatar may be controlled by using various methods, such as displaying thickly the dominant foot, representing running/walking/limping as the movement of the avatar, or changing the display in response to changes in weight or walking intensity.

In addition, according to another embodiment, the present disclosure may further realize an interaction function through treadmill control in providing a content-linked service.

Specifically, the present disclosure may be implemented to adjust the inclination angle of the treadmill 10 based on gait-related content provided by the content-linked service, and there may be various methods by which the present disclosure is implemented.

Accordingly, the controller 130 may provide an interaction function through treadmill control by analyzing changes in exercise characteristics due to the adjustment of the inclination angle of the treadmill 10 from gait data generated from the treadmill 10 and providing feedback on an optimal inclination angle of the treadmill 10.

Furthermore, the present disclosure may provide the function of using a speech recognition function included in a device, such as the smartphones 30 or the display devices 40, that is positioned close to a user walking on the treadmill 10, to directly convert or automatically translate conversations during walking into text, thereby enabling the user to interactively control the treadmill 10 or content-linked services, or enabling the user to communicate with another person whom the user meets in the virtual space via content to be described later and shares interaction in the virtual space.

In addition, the present disclosure may further provide a function of simple psychological state diagnosis and feedback (initially providing a fortune cookie concept, but after data collection, providing precise diagnostic information) based on gait analysis, as well as providing a gait analysis-based content-linked service. To this end, a process of entering simple personal information (e.g., age, height, weight, gender, etc.) in advance (no personally identifiable information such as a name is required).

As described above, in the present disclosure, a gait analysis-based content-linked service may be provided based on gait data generated for walking using the existing treadmill 10, and the generated gait data may be transmitted to a linkage service (e.g., medical institutions, various DBs, etc.) while providing the content-linked service, so as to be used for various gait analysis technologies (e.g., CNN, ensemble model, etc.).

Accordingly, the present disclosure provides a gait analysis-based content-linked service, which uses a change in gait pattern according to gait analysis as a control input for content, thereby helping the user to perform walking exercises on the treadmill 10 without getting bored. This enables more gait data to be obtained, and may result in the development of various types of content and the growth of services that can be combined with the treadmill 10. Particularly, in the case of content for an artwork exhibition hall, an exhibited artwork may be provided with an orientation and then allowed to be actually viewed, thereby increasing the understanding of the artwork and the exhibition.

In the present disclosure, information about gait analysis obtained while providing a gait analysis-based content-linked service may be transmitted to a relevant medical institution for use in monitoring long-term disease conditions at home, and thus may also be used in an analysis-based disease diagnosis service.

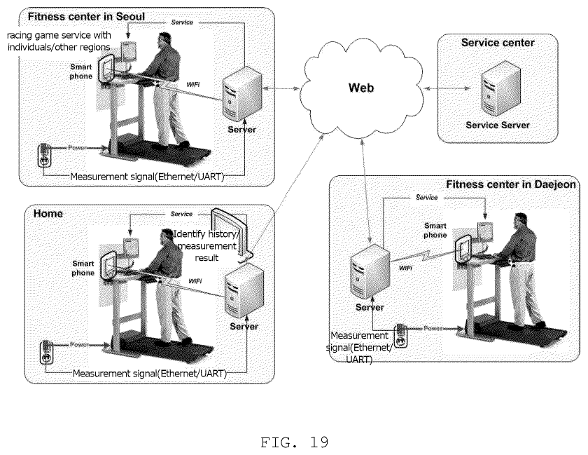

Furthermore, FIG. 19 illustrates an example of a shared interaction in a virtual space that can be provided by the gait analysis-based content-lined service of the present disclosure.

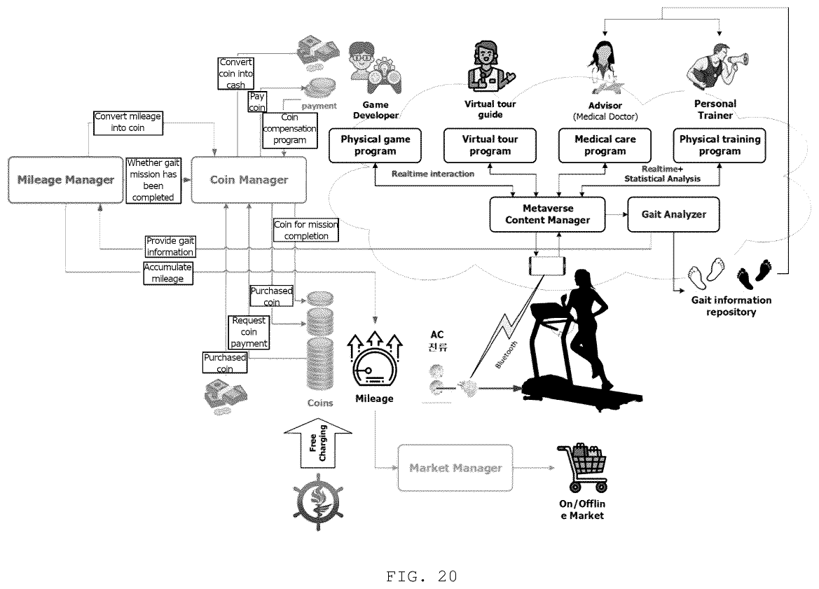

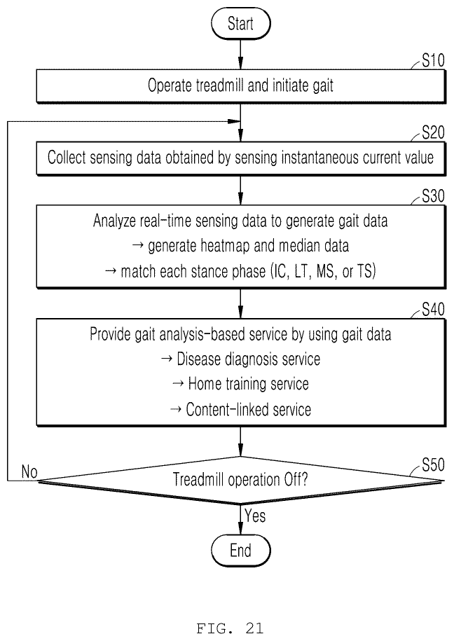

Furthermore, as illustrated in FIG. 20, various service methods based on gait analysis may be provided according to embodiments of the present disclosure.

As described above, various business models may be derived based on a gait analysis-based service method according to embodiments of the present disclosure.

As illustrated in FIG. 19, the present disclosure may provide a gait analysis-based content-linked service, which is combined with shared interaction in a web-based virtual space, by using gait data for multiple persons that can be obtained from multiple treadmills in the same place or different places.

In this way, multiple persons in the same place or different places can meet virtually through content that provides shared interaction in a virtual space, and use a gait analysis-based content-linked service that uses their gait patterns as control inputs for the content.

As described above, the present disclosure implements a specific technical solution for quantitatively and objectively analyzing walking on an existing treadmill by using an adapter sensor that can be attached to the motorized treadmill in a simple manner without modification.

Accordingly, the present disclosure may realize an environment in which quantitative/objective gait analysis results can be obtained in the form of lifelog data without the need for complex and expensive equipment, thereby enabling the provision of various services (e.g., disease diagnosis services, home training services, content-linked services, and the like) based on gait analysis results, such as enabling disease prediction in the concept of precision medicine.

As a business model using the gait analysis device 100 and a service method based on gait analysis according to an embodiment of the present disclosure, as illustrated in FIG. 20, information may be transmitted through a network between the gait analysis device 100, a mileage manager, a coin manager, a metaverse content manager, and a market manager, and various services may be provided.

Each of the mileage manager, the coin manager, the metaverse content manager, and the market manager may be a separate server or terminal.

To provide services according to an embodiment of the present disclosure, a game developer, a virtual travel guide, a doctor, a trainer, etc. may be connected to the metaverse content manager through their respective separate terminals.

A game program may be transmitted to the metaverse content manager and a user's terminal (e.g., the smartphone 30) through the game developer's terminal, and the user may respond via a treadmill in real time. Thus, a game based on gait information may be played.

A travel program may be transmitted to the metaverse content manager and the user's terminal (e.g., the smartphone 30) through the virtual travel guide's terminal, and the user may respond through the treadmill in real time. Thus, the virtual travel based on gait information may be performed.

A physical training program may be transmitted to the metaverse content manager and the user's terminal (e.g., the smartphone 30) through the trainer's terminal, and the user may respond via the treadmill in real time. Thus, a physical training program based on the gait information may be executed.

A medical care program may be transmitted to the metaverse content manager and the user's terminal (e.g., the smartphone 30) through the doctor's terminal, and the user may respond via the treadmill in real time. Thus, medical services based on gait information may be provided.

The user may use virtual currency for the use of each service (e.g., game, virtual travel, disease diagnosis, home training, content-linked service, etc.), and virtual currency may be transmitted to each service provider through the coin manager.

When the user walks on a treadmill by a gait analysis-based service according to an embodiment of the present disclosure, mileage may be accumulated for the user according to each piece of gait information.

The accumulated mileage may be converted into coins through the coin manager.

The accumulated mileage may be used to purchase goods offline through the market manager.

Hereinafter, the flow of operations of a gait analysis device according to an embodiment of the present disclosure will be described in detail with reference to FIG. 21.

For convenience, a description will be made using the reference numerals disclosed in FIGS. 2 and 3.

When the treadmill 10 is operated, the sensor 20 senses an instantaneous current value caused by a change in motor load of the treadmill 10 to monitor a change in the instantaneous current value due to the change in the motor load in real time (S10)

When the treadmill 10 is operated and when a person (a user) starts walking on the treadmill 10, the sensor 20 senses an instantaneous current value caused by a change in motor load of the treadmill 10, and monitors a change in the instantaneous current value due to the change in the motor load caused by the user's walking in real time (S10).

When the treadmill 10 starts to operate (S10), the gait analysis device 100 according to an embodiment of the present disclosure may collect real-time sensing data, sensed by the sensor 20, by using various transmission methods (S20).

Furthermore, when a person (the user) starts walking on the treadmill 10 (S10), the gait analysis device 100 according to an embodiment of the present disclosure may collect real-time sensing data sensed by the sensor 20 by using various transmission methods (S20).

The gait analysis device 100 according to an embodiment of the present disclosure analyzes the collected sensing data to generate gait data divided into gait posture phases (stance phase) defined by a specific vision-based reference model (S30). As described above, various types of gait data may be generated by a data generator.

In the present disclosure, as the specific vision-based reference model, an existing model that distinguishes between gait posture phases (stance phase) for a step may be adopted and used. However, in the following embodiments, the RLA gait phase model will be described as a specific reference model.

More specifically, the gait analysis device 100 may extract individual steps from continuous outer envelopes extracted from sensing data, and may overlap data regarding the extracted individual steps to generate a heatmap in which data for each step is distributed for each distinction time point within a unit time.

A unit time refers to one second, and a distinction time point within a unit time may be related to a sampling period at which sensing data is sensed (e.g., 1/60 seconds for 60 Hz AC current, and 1/50 seconds for 50 Hz AC current).

That is, the gait analysis device 100 may overlap data of individual steps extracted from the sensing data (changes in instantaneous current value) to generate a heatmap in which the distribution of data for each step is visually shown at intervals of 1/60 seconds as the steps overlap.

Furthermore, the gait analysis device 100 may derive a median graph from the sensing data (changes in instantaneous current value)

The gait analysis device 100 may generate gait data by matching each gait posture phase (e.g., IC, LR, MS, or TS) defined in the above-described specific reference model (e.g., the RLA gait phase model) to at least one of the heatmap and the median graph.

Thus, the present disclosure realizes features wherein changes in load of the motor of the treadmill 10 depending on the gait of a person (a user), i.e., instantaneous current values, are sensed by using the sensor 20 attachable to the treadmill 10 in a simple manner without modification; and gait data (a heatmap and a median graph) to which each gait posture phase (e.g., IC, LR, MS, or TS) defined by the RLA gait phase model is matched is generated from the sensing data.

Accordingly, the gait analysis device 100 according to an embodiment of the present disclosure may provide various gait analysis-based services by using the gait data (the heatmap and the median graph) generated in operation S30, based on linkage with various linkage services (e.g., medical institutions, content servers, various DBs, etc.) outside the gait analysis device 100 (S40).

In one example, the gait analysis device 100 may provide a disease diagnosis service based on gait analysis by using gait data (the heatmap and the median graph), and examples of providing the disease diagnosis service have been described in detail with reference to FIGS. 10 to 14 above, and thus will be omitted herein.

In another example, the gait analysis device 100 may provide a home training service based on gait analysis by using gait data (the heatmap and the median graph), and examples of providing the home training service have been described in detail with reference to FIGS. 15 to 17 above, and thus will be omitted herein.

In another example, the gait analysis device 100 may provide a gait analysis-based content-linked service by using gait data (the heatmap and the median graph), and examples of providing the content-lined service have been described in detail with reference to FIGS. 18 and 19 above, and thus will be omitted herein.

On the basis of the realization of an environment in which quantitative/objective gait analysis results of an individual can be obtained in the form of lifelog data by using the existing treadmill 10 without complex and expensive equipment, the gait analysis device 100 according to an embodiment of the present disclosure may perform gait analysis by combining the present disclosure with various existing visual gait analysis technologies and numerical/quantitative gait analysis technologies, and may provide a new gait analysis-based service.

Furthermore, as long as the operation of the treadmill 10 is not turned off (S50 No), the gait analysis device 100 may continue collecting sensing data to perform gait data generation and service provision.

As can be seen from the above detailed description, the present disclosure implements a specific technical solution for quantitatively and objectively analyzing walking on an existing treadmill by using an adapter sensor that can be attached to a motorized treadmill in a simple manner without modification.

Accordingly, the present disclosure may realize an environment in which quantitative/objective gait analysis results can be obtained in the form of lifelog data without the need for complex and expensive equipment, thereby enabling the provision of various services (e.g., disease diagnosis services, home training services, content-linked services, and the like) based on gait analysis results, such as enabling disease prediction in the concept of precision medicine.

The operation method of the gait analysis device according to one embodiment of the present disclosure, described above, may be implemented in the form of a program command that can be executed through various computer means, and may be recorded on a computer-readable medium. The computer-readable medium may include program commands, data files, data structures, etc., alone or in combination. The program commands recorded in the medium may be specifically designed and configured for the present disclosure or may be known and available to those skilled in the art of computer software. Examples of computer-readable recording medium include: magnetic media, such as hard disks, floppy disks, and magnetic tape; optical media, such as CD-ROMs and DVDs; magneto-optical media, such as floptical disks; and hardware devices specifically configured to store and execute program commands, such as ROMs, RAMs, and flash memory; and virtual cloud storage, which is connected to a network and distributed/stored in specific storage devices. Examples of program commands include machine language code, which is produced by a compiler, as well as high-level language code that may be executed by a computer using an interpreter or the like. The aforementioned hardware devices may be configured to operate as one or more software modules to perform the operations of the present disclosure, and vice versa.

Although the present disclosure has been described in detail with reference to exemplary embodiments, the present disclosure is not limited to the above embodiments, and those skilled in the art to which the present disclosure belongs will understand that various variations or modifications can be derived from the technical idea of the present disclosure without departing from the subject matter of the present disclosure as claimed in the following claims.

What is claimed is:

1. A gait analysis device comprising:
a data collector configured to collect sensing data obtained by sensing an instantaneous current value that is supplied to a treadmill to operate a motor of the treadmill;
a data generator configured to analyze the sensing data to generate a user's gait data; and
a controller configured to send the gait data so that the gait data is displayed, recorded, or transmitted,
wherein the data generator is configured to:
set a threshold that is a maximum value of the instantaneous current value for a predetermined time in an idle state; and
generate the gait data by analyzing the sensing data from a time point when the instantaneous current value exceeds the threshold.

2. The gait analysis device of claim 1, wherein the data generator is configured to:
extract a first step and a second step of the user by continuous outer envelopes extracted from the sensing data, the first step and the second step being repeated sequentially; and
distinguish a step of the user's left foot from a step of the user's right foot by defining a series of the first steps as index walking in case that maximum values of envelopes of the series of the first steps are greater than values obtained by adding a reference value to maximum values of envelopes of a series of the second steps.

3. The gait analysis device of claim 1, wherein the data generator is configured to analyze the collected sensing data to generate the gait data, which is divided into gait posture phases (stance phase) defined in a specific vision-based reference model.

4. The gait analysis device of claim 1, wherein the data generator is configured to:
extract the user's step by using a continuous outer envelope extracted from the sensing data; and
generate the gait data by distinguishing, in the envelope constituting the one step, an LR section in which a current value increases, an MS section in which the current value decreases after the LR section, and a PW section in which the current value decreases and reaches a minimum value after the MS section.

5. The gait analysis device of claim 4, wherein the data generator is configured to distinguish, in the envelope constituting the one step, a TS section in which the current value decreases after the MS section, wherein the TS section is before the PW section, and a decreasing slope of the TS section is less than a decreasing slope of the MS section and a decreasing slope of the PW section.

6. The gait analysis device of claim 3, wherein the data generator is configured to:
extract individual steps from continuous outer envelopes extracted from the sensing data;
overlap data of the extracted individual steps to generate a heatmap in which data for each step is distributed at each distinction time point within a unit time; and
generate the gait data by matching each gait posture phase defined in the specific vision-based reference model to at least one of the heatmap and a median graph derived from the sensing data.

7. The gait analysis device of claim 4, wherein the data generator is configured to:
extract individual steps from continuous outer envelopes extracted from the sensing data;
overlap data of the extracted individual steps to generate a heatmap in which data for each step is distributed at each distinction time point within a unit time; and
generate the gait data by matching each gait posture phase defined in the specific vision-based reference model to at least one of the heatmap and a median graph derived from the sensing data.

8. The gait analysis device of claim 5, wherein the data generator is configured to:
extract individual steps from continuous outer envelopes extracted from the sensing data;
overlap data of the extracted individual steps to generate a heatmap in which data for each step is distributed at each distinction time point within a unit time; and
generate the gait data by matching each gait posture phase defined in the specific vision-based reference model to at least one of the heatmap and a median graph derived from the sensing data.

9. The gait analysis device of claim 6, wherein the controller is configured to use at least one of the heatmap and the median graph as the gait data to: analyze a gait control state based on a distribution degree;
or analyze a predicted disease based on at least one among the shape of a curve, a distinction location for each gait posture phase, a slope between the distinction locations, and relative sizes of the distinction locations.

10. The gait analysis device of claim 3, wherein the controller is configured to calculate the degree of pain and discomfort related to the user's predicted disease by additionally reflecting the collected sensing data in the analysis in case that the predicted disease has been analyzed using the gait data.

11. The gait analysis device of claim 4, wherein the controller is configured to calculate the degree of pain and discomfort related to the user's predicted disease by additionally reflecting the collected sensing data in the analysis in case that the predicted disease has been analyzed using the gait data.

12. The gait analysis device of one of claim 5, wherein the controller is configured to calculate the degree of pain and discomfort related to the user's predicted disease by additionally reflecting the collected sensing data in the analysis in case that the predicted disease has been analyzed using the gait data.

13. The gait analysis device of claim 3, wherein the controller is configured to:
provide gait-related content to a display device of a user walking on the treadmill; and
control the providing of the gait-related content to the display device based on real-time gait patterns and gait pattern changes analyzed using the gait data.

14. The gait analysis device of claim 4, wherein the controller is configured to:
provide gait-related content to a display device of a user walking on the treadmill; and
control the providing of the gait-related content to the display device based on real-time gait patterns and gait pattern changes analyzed using the gait data.

15. The gait analysis device of claim 5, wherein the controller is configured to:
provide gait-related content to a display device of a user walking on the treadmill; and
control the providing of the gait-related content to the display device based on real-time gait patterns and gait pattern changes analyzed using the gait data.
